(19) United States
(12) Patent Application Publication
(10) Pub. No.: US 2025/0237083 A1
(43) Pub. Date: Jul. 24, 2025

(54) SMART SELF-DISINFECTING DOOR HANDLE/DOOR PULL

(71) Applicant: UVEFX LLC, Calabasas, CA (US)

(72) Inventors: Ashkan Okhovat, Calabasas, CA (US); Vladimir Factor, Calabasas, CA (US)

(21) Appl. No.: 18/418,256

(22) Filed: Jan. 20, 2024

(51) Int. Cl. E05B 1/00; A61L 2/10

(52) U.S. Cl. CPC E05B 1/0069; A61L 2/10; A61L 2202/11; A61L 2202/121; A61L 2202/14; E05B 1/003

(57) ABSTRACT

A self-disinfecting door handle device which uses a variety of mechanisms to sanitize the handle via UV light. One such mechanism uses light passable materials for an internal UV light to disinfect the exterior of the handle. Another such mechanism uses a magnetically controlled outer ring to safely disinfect the exterior of the door handle without exposing individuals to the UV light. An internal rail system magnetically pulls the outer ring the length of the handle. The internal rail system is connected to a power source inside the door panel. The outer ring has a wirelessly rechargeable battery.

### BACKGROUND

### Technical Field

The present disclosure generally relates to devices that inhibit the spread of viruses, bacteria, mold, yeasts, pathogens. More particularly, the present disclosure is related to a self-disinfecting door handle/door pull.

### Description of the Related Art

The bacteria and viruses on fomite surfaces such as door handles, doorknobs, door pulls, etc., can spread viruses, bacteria, mold, and yeasts to other frequently touched objects at home or work.

### SUMMARY

In one embodiment, a self-disinfecting door handle device includes a first door handle having a first grip portion constructed of an ultraviolet (UV) light passable material having at least a semi-transparency for passage of an ultraviolet light. A latch assembly is rotatably connected to the first door handle, the latch assembly includes a latch that is configured to retract according to a rotated of the first door handle. A UV lamp is arranged within the first door handle, and includes a connector configured to electrically connect to a UV light source that emits a UV light toward a surface of the first door handle.

In an embodiment, the UV map emits light having a wavelength of 222 nm.

In an embodiment, the latch assembly includes a battery compartment electrically connected to the UV lamp, and the UV lamp is receiving power from a power source arranged in the battery compartment selected from the group consisting of lithium ion, sodium ion, potassium ion, lithium-sulfur, sodium sulfur, potassium sulfur, and graphene.

In an embodiment, the UV light source is electrically connected to the UV lamp, and the UV light source comprises a plug and play UV tube.

In an embodiment, the UV light source is a ballast-free UV light source.

In an embodiment, the UV light source is one or more UV light emitting diodes (LED).

In an embodiment, the first door handle is a substantially tubular shape configured to enclose the UV light source. In another embodiment, the door handle is substantially cubical in shape.

In an embodiment, a second door handle is rotatably connected to an opposite side of the latch assembly, the second door handle having a second grip portion constructed of an ultraviolet (UV) light passable material having at least a semi-transparency for passage of an ultraviolet light.

In an embodiment, the first door handle and/or the second door handle is coated with an anti-microbial substance.

In an embodiment, at least the grip of the first door handle and/or the grip of the second door handle is constructed of an anti-microbial material.

In an embodiment, the UV light source emits UVC light toward an exterior surface of the first grip portion of the first door handle.

In an embodiment, the UV light source emits FAR-UV light toward an exterior surface of the first grip portion of the first door handle.

In an embodiment, the UV light source emits NEAR-UV light toward an exterior surface of the first grip portion of the first door handle.

In an embodiment, a sensor is arranged in the first door handle that is configured to detect when the first grip portion handle is contacted by a user.

In an embodiment, the sensor activates the UV light source based on whether the sensor detects a contact of the first grip portion. The dosage of FAR-UV can be varied based on applicable rules and regulations.

In one embodiment, a self-disinfecting door pull includes a handgrip configured for attachment to a door. A sensor is configured to detect contact with an exterior surface of the handgrip, and an ultra-violet (UV) light source is arranged to emit UV light at the exterior surface of the handgrip.

In an embodiment, an exterior of the handgrip is constructed of an anti-microbial material and/or coated with an anti-microbial agent.

In an embodiment, the UV light is a UV LED light arranged external to the door pull.

In an embodiment, the UV light source is a ballast-free UV light source arranged within the handgrip, and the handgrip is constructed of a UV-passable material.

In an embodiment, the UV light source is configured to emit UV light at a surface of the handgrip for a predetermined period of time after the sensor detects contact with the exterior surface of the tube.

In an embodiment, the UV light source is configured to emit UV light at the handgrip for predetermined intervals of time.

In an embodiment, a latch assembly is rotatably connected to the handgrip, and the latch assembly includes a battery compartment configured for receiving a lithium battery that powers the UV light source.

In an embodiment, where there is an outer ring on the door handle with a 254 nm UV germicidal light inside of the outer ring. The outer ring seals the UV light inside the outer ring to limit exposure to only the handle and not people who might be in the room.

The outer ring is moved along the exterior of the door handle based on an inner magnet that is mounted on a rail system in the interior of the handle. As the interior magnet moves through the handle, the outer ring where the UV light is located moves along the surface of the handle, disinfecting the surface. The end of the handle has a cap or ridge which prevents the outer ring from accidentally coming off.

The interior rail system is powered directly by wires from the battery located inside the doorplate. The exterior ring where the UV light is located has a battery that is wirelessly charged while not in use and located in the interior position.

These and other features will become apparent from the following detailed description of illustrative embodiments thereof, which is to be read in connection with the accompanying drawings.

### BRIEF DESCRIPTION OF THE DRAWINGS

The drawings are of illustrative embodiments. They do not illustrate all embodiments. Other embodiments may be used in addition to or instead. Details that may be apparent or unnecessary may be omitted to save space or for more effective illustration. Some embodiments may be practiced with additional components or steps and/or without all the components or operations that are illustrated. When the same numeral appears in different drawings, it refers to the same or like components or operations. It is to be understood by a person of ordinary skill in the art that none of the drawings of the self-disinfecting door handle are drawn to scale.

FIG. 1 shows a perspective view of a self-disinfecting door handle according to an embodiment of the present disclosure.

FIG. 2A shows a perspective rear view of self-disinfecting door handle according to an embodiment of the present disclosure.

FIG. 2B shows detail regarding a twist lock and an indicator lights on the inside handle and the outside handle according to an embodiment of the present disclosure.

FIG. 3 is an exploded front perspective view of the self-disinfecting door handle according to an embodiment of the present disclosure.

FIG. 4 is a front view of one type of handle according to an embodiment of the present disclosure.

FIG. 5 is a rear view of the self-disinfecting door handle according to an embodiment of the present disclosure.

FIG. 6 is a top view show of the self-disinfecting door handle attached to a portion of a door according to an embodiment of the present disclosure.

FIG. 7 is a bottom view show of the self-disinfecting door handle attached to a portion of a door according to an embodiment of the present disclosure.

FIG. 8 is one side view show of the self-disinfecting door handle attached to a portion of a door according to an embodiment of the present disclosure.

FIG. 9 is another side view opposite the view shown in FIG. 8 of the self-disinfecting door handle attached to a portion of a door according to an embodiment of the present disclosure.

FIG. 10A is view of the ultraviolet lamp assembly of the self-disinfecting door handle according to an embodiment of the present disclosure.

FIG. 10B is an exploded view of the ultraviolet lamp assembly of the self-disinfecting door handle shown in FIG. 10A according to an embodiment of the present disclosure

FIG. 11 is an exploded view of some components of the door handle according to an embodiment of the present disclosure.

FIG. 12A is a side view of a self-disinfecting door handle grasped to open/close a door according to an embodiment of the present disclosure.

FIG. 12B shows a plurality of projections of the ultraviolet light from a self-disinfecting door handle grasped to open/close a door according to an embodiment of the present disclosure.

FIG. 12C is a front view of a self-disinfecting door handle grasped to open a door according to an embodiment of the present disclosure.

FIG. 13 is a perspective view of the internal battery system according to an embodiment of the present disclosure.

FIG. 14 is a front view of a self-disinfecting door pull according to an embodiment of the present disclosure.

FIG. 15 is a perspective front view of a self-disinfecting door pull according to an embodiment of the present disclosure.

FIG. 16 is a perspective rear view of a self-disinfecting door pull according to an embodiment of the present disclosure.

FIG. 17 is a perspective rear view of a self-disinfecting door pull according to an embodiment of the present disclosure.

FIG. 18 is a perspective front view of a self-disinfecting door pull according to an embodiment of the present disclosure.

FIG. 19 is a perspective front view of a self-disinfecting door handle according to an embodiment of the present disclosure.

### DETAILED DESCRIPTION

### Overview

In the following detailed description, numerous specific details are set forth by way of examples to provide a thorough understanding of the relevant teachings. However, it should be understood that the present teachings may be practiced without such details. In other instances, well-known methods, procedures, components, and/or circuitry have been described at a relatively high level, without detail, to avoid unnecessarily obscuring aspects of the present teachings.

### CONCLUSION

The descriptions of the various embodiments of the present teachings are presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

The components, operations, steps, features, objects, benefits, and advantages that have been discussed herein are merely illustrative. None of them, nor the discussions relating to them, are intended to limit the scope of protection. While various advantages have been discussed herein, it will be understood that not all embodiments necessarily include all advantages. Unless otherwise stated, all measurements, values, ratings, positions, magnitudes, sizes, and other specifications that are set forth in this specification, including in the claims that follow, are approximate, not exact. They are intended to have a reasonable range that is consistent with the functions to which they relate and with what is customary in the art to which they pertain.

There are also embodiments of the disclosure in which the components and/or operations are arranged and/or ordered differently than described and shown herein.

It will be understood that the terms and expressions used herein have the ordinary meaning as is accorded to such terms and expressions with respect to their corresponding respective areas of inquiry and study except where specific meanings have otherwise been set forth herein. Relational terms such as first and second and the like may be used solely to distinguish one entity or action from another without necessarily requiring or implying any such actual relationship or order between such entities or actions. The terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element proceeded by “a” or “an” does not, without further constraints, preclude the existence of additional identical elements in the process, method, article, or apparatus that comprises the element. It is to be understood that the appended claims are not limited to the embodiments shown herein.

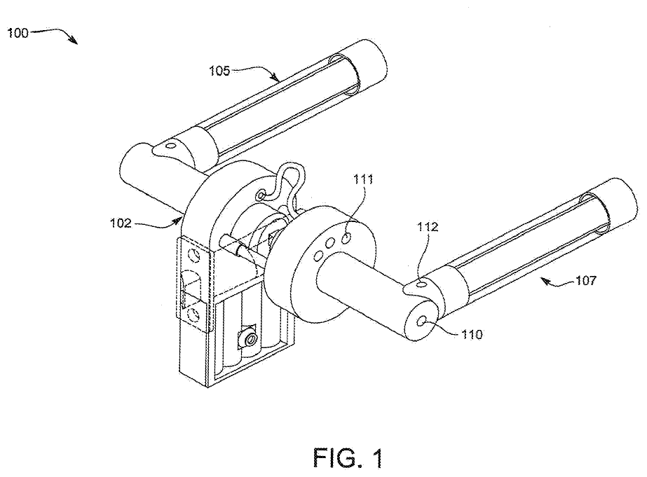

FIG. 1 shows a perspective view 100 of a smart self-disinfecting door handle according to an embodiment of the present disclosure. It is to be understood that the claimed subject matter of the present disclosure is not limited to door handle or door pulls. There is shown two handles 105,107 that are connected to a door handle assembly 102. The door handles 105,107 may be constructed of an anti-microbial material. Alternatively, an anti-microbial coating may be applied to the door handles 105, 107. For illustrative purposes the door handle 105 may be referred to as an “inside door handle: and the door handle 107 may be referred to as an “outside door handle”. It is to be understood that these door handle terms may be the opposite. Also shown is a safety motion sensor 110, indicator lights 111, and a grip twist lock 112. It is to be understood that the aforementioned components enhance the operation of device, as discussed at least in the next paragraph. However, the claimed subject matter of the present disclosure is not limited by these components, some or all of which are optional.

The safety motion sensor 110 senses when someone comes close to the door, and sends a signal to interrupt a sanitizing cycle and turn off the UV lights.

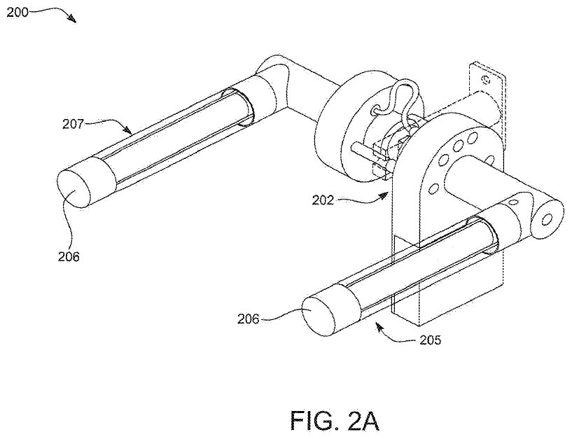

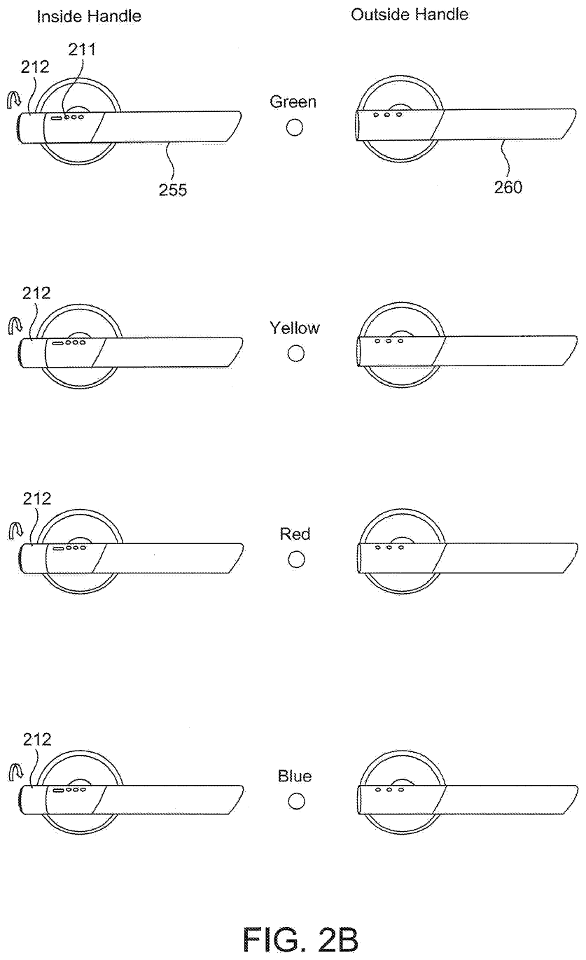

The indicator lights 111 are configured for a variety of different purposes. For example, the lights may have green, yellow, red and blue colors that are emitted to indicated various information. It is to be understood that the claimed subject matter is not limited to the quantity, color, arrangement, or type of indications discussed herein for illustrative purposes. Still referring to FIG. 1, and with reference to FIG. 2B, regarding the inside door handle 105/255, when the door is unlocked and someone comes within a predetermined distance (e.g., 1 foot) of the handle, a green indicator light 111/211 turns on indicating that the door is unlocked, no one is standing behind it, and the UV light is off, and safe to touch. When a dial on the grip twist lock 112/212 is rotated to unlock the door, the green indicator light 111/211 turns on for the predetermined time period (e.g. 2-5) seconds to let you know that the door is unlocked. The dial on the grip twist lock 112/212 can be rotated to a middle yellow level and the yellow indicator in the middle turns on for a predetermined time period (e.g. 2-5 seconds), confirming that the handle is on a non-sanitizing mode, and the UV light is off. When the dial is rotated from inside to lock the door, the indicator lights 111/211 will turn on for the predetermined time period (e.g. 2-5 seconds), confirming that the door is locked. In this embodiment, the indicator setting is the same on both sides of the door. The predetermined time period can be different for various functions described above.

With regard to the outside door handle 107/260, as a person approaches within the predetermined distance (e.g. 1 foot), all of the indicator lights 111 may blink yellow from outside when the door is unlocked and there is someone standing behind the door.

When the door is locked a phone or a finger print scanner, a red indicator light 111 confirms the door is locked.

When someone from outside approaches the door, and the door is locked, all of the indicators may turn red for a few seconds. After every touch to the door handle, the indicator lights may turn blue and the UV light inside the tinted glass handle will turn on to sanitize the glass, and turn off after 30 seconds. It is to be understood that the time the UV light is turned off is provided for illustrative purposes, and may be less than or greater than 30 seconds.

With continued reference to FIG. 1, the door handles 105, 107 are constructed of a material that permits the passage of ultraviolet (UV) light. The door handles 105, 107 may also be constructed of the anti-microbial material. Different materials may be used depending upon the type of UV light used. For example, Far-UV, Near-UV are two additional types of UV light that can be used, and the types of materials used for the handles can differ based on the type of UV light. In addition, in an embodiment, a Far UV of 222 nm (which some studies have shown to be as much as 100 times safer than UV 254 nm light) is used to enhance user safety, as this wavelength is being studied as a way to reduce any potential effects on the skin. Near-UV light is more effective for disinfection against, for example, bacteria and certain pathogens, but less effective or possibly non-effective against certain viruses. Thus, the claimed subject matter of the present disclosure contemplates that the wavelength selection of bands of UV light, and/or the application of Far-UV light, UV light, or near-UV light may be used for specific types of disinfection, and/or for a broad range of disinfection. Materials such as highly purified calcium fluoride (CaF₂) can be used for applications of light down to about 160 nm. In addition, MgF₂, Lithium Fluoride (LIF) are substantially UVC to about 110 nm. Borate crystals and silica (both UV grade 200 nm and standard grade 260 nm) can provide UV transparency in various degrees. Filters may be used to enhance user safety at certain wavelengths of UV light (as well as Far-UV, near-UV, etc.).

In an embodiment of the present disclosure, UV-C light is particularly effective for disinfection of certain microbial agents. More particularly, for certain viruses and bacteria, a range of 200 nm to 280 nm, or sub-ranges of this range, can be used. It is to be understood that the claimed subject matter is not limited to any particular type of organism/pathogen. For example, a smart self-disinfecting device according to the present disclosure is effective against wide variety of bacteria, viruses, fungus, pathogens, molds, fungus, etc., just to name a few non-limiting applications. The self-disinfecting device can also be used for more specific types of disinfection directed to a particular type of virus, bacteria fungus, pathogen, mold fungus, or some of the aforementioned.

The door handles 105, 107 are touched by successive persons who partially rotate the door handles 105, 107 to retract a latch (shown in the dotted lines) that is typically positioned within an opening in a strike plate in the doorframe. For example, there may be a single self-disinfecting door handle attached to a “pull-side” of a door, is referred to as a “pull handle”, and is often used for an inner door handle within an office, a restaurant, or other type of commercial venue. Such pull handles may not be partially rotatable, and may not have a latch assembly, or control the operation of the latch assembly.

With continued reference to FIG. 1, the appended claims are not limited to the tubular shape of two handles 105, 107 shown. In this particular embodiment, the shape of the handles 105, 107 is similar to the shape of the ultraviolent lamps connected within the handles. However, the handles may be embodied in other shapes (e.g., rectangular, flat, etc.).

FIG. 2A shows a perspective rear view 200 of a self-disinfecting door handle according to an embodiment of the present disclosure. The handles 205, 207 are shown in a leftward orientation. A read side of the latch assembly 202 is shown. The dashed lines show a rear of a latch that would extended into an opening in a strike plate. A fingerprint scanner 206 is shown in the door handles.

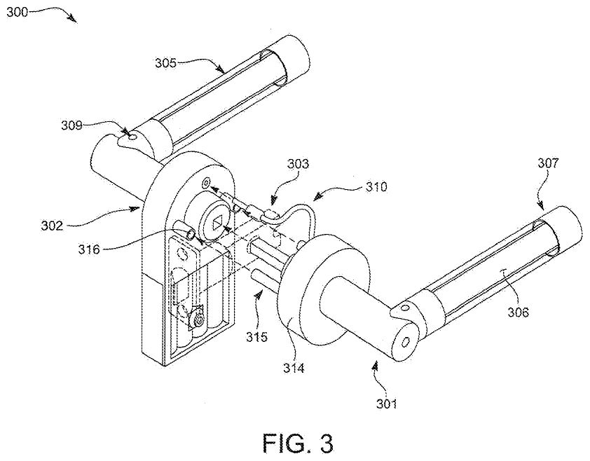

FIG. 3 is an exploded front perspective view 300 of the self-disinfecting door handle according to an embodiment of the present disclosure. Similar to the embodiment in FIG. 1, there are two tubular-shaped handles 305, 307. The handle assembly 301 includes an ultraviolet (UV) bulb socket configured to receive a UV lamp. It is also to be understood that the UV lamps according to the present disclosure, are ballast-free lamps. The ballast-free construction provides for applications in which a reduced bulk and power usage are factors in the construction. The engagement portion 314 includes protrusions 315 that are sized to be received by sleeves 316 in the latch assembly 302. Alternatively, recesses may be used instead of sleeves 316.

As previously discussed, the handles 305, 307 are constructed of a translucent material so that the ultraviolet light output by the UV bulb 306 will disinfect the surfaces of the handles 305, 307. A wire 310 connects the UV bulb 306 to a power connector in the latch assembly 302. In an embodiment, the power connector is configured to receive power from a power source, such as, for example, lithium batteries. Each of the handles has an opening 309 that houses a sensor that sensors whether the handle has been grabbed.

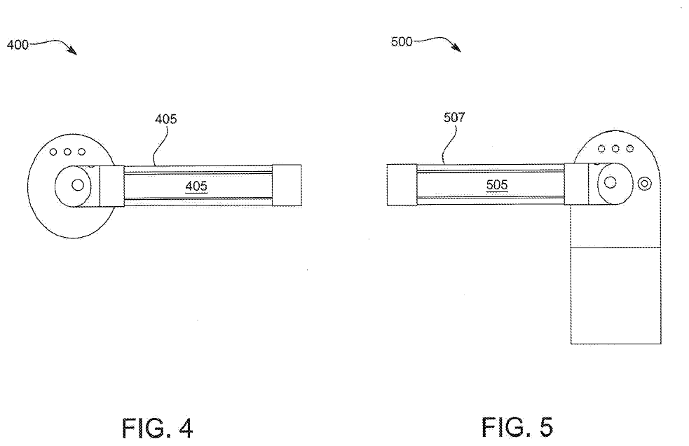

FIG. 4 is a front view 400 of one type of handle 405 according to an embodiment of the present disclosure. The handle has a UV bulb 406 within.

FIG. 5 is a rear view of the self-disinfecting door handle 507 according to an embodiment of the present disclosure. The handle has an ultraviolet bulb 505 within is similar to the ultraviolet light shown in FIG. 4.

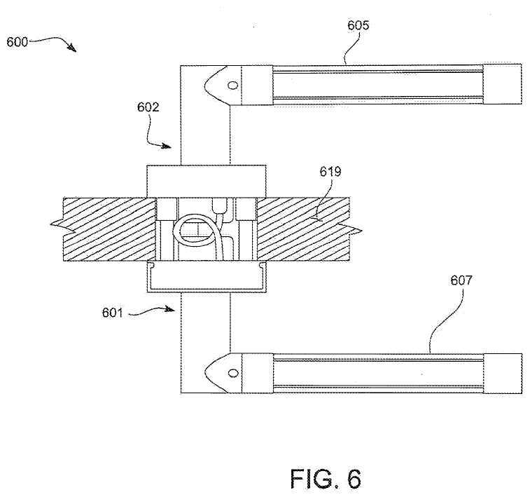

FIG. 6 is a top view 600 showing the self-disinfecting door handle attached to a portion of a door according to an embodiment of the present disclosure. The door handle is attached to a door 619. It can be seen that the attachment portion 601, 602 of the door 619. The handles 605, 607 are similar to the handles shown in FIG. 1. However, the present disclosure is not limited to the handles shown in FIG. 6.

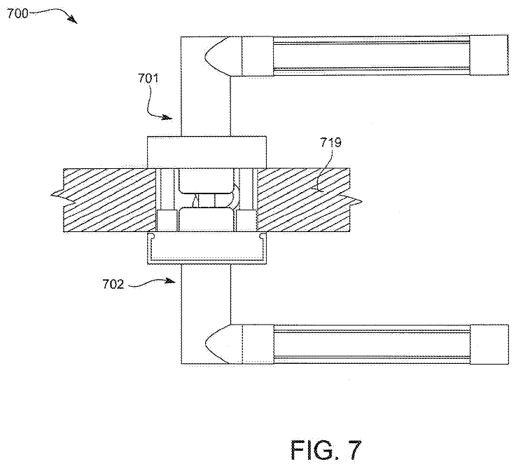

FIG. 7 is a bottom view 700 showing of the self-disinfecting door handle attached to a portion of a door 719 according to an embodiment of the present disclosure. The attachment portions 701, 702 are arranged on opposite sides of the door 719. In viewing FIG. 6 and FIG. 7, it can be seen that the door handle assembly does not require the door to be wired as a conventional UV light. For example, one or more lithium batteries can be used to fit within the door assembly and provide power for the UV lights.

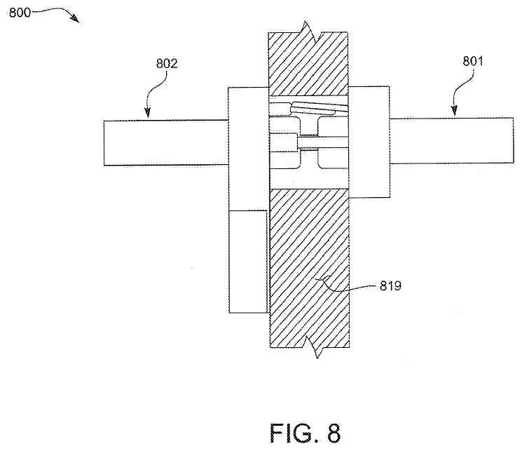

FIG. 8 is one side view show of the self-disinfecting door handle attached to a portion of a door according to an embodiment of the present disclosure. FIG. 8 shows a portion of a door 819 in its normal orientation, and the door handle assembly attached to the door via an opening. The door handles 801, 802 are arranged at opposites sides of the door. It is also to be understood that other shapes, including a knob can be used. There are mechanical connections such as the previously-discussed (see the description of FIG. 3 above) protrusions and sleeves, and electrical connections from the power source in the latch assembly to the UV lamps in both door handles 801, 802.

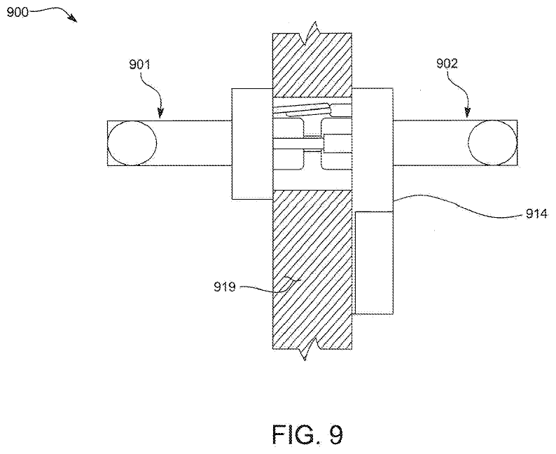

FIG. 9 is another side view opposite the view shown in FIG. 8 of the self-disinfecting door handle attached to a portion of a door. The latch assembly 914 is shown on the right side of the door.

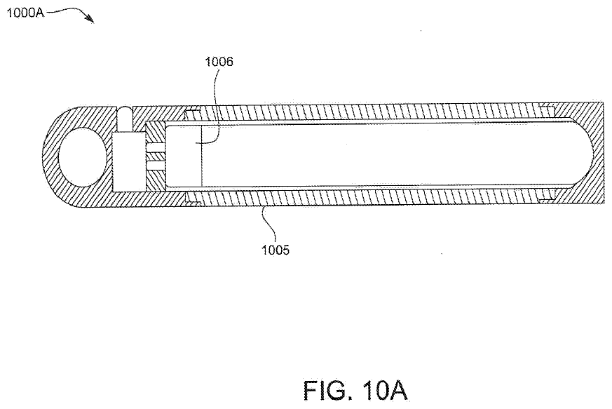

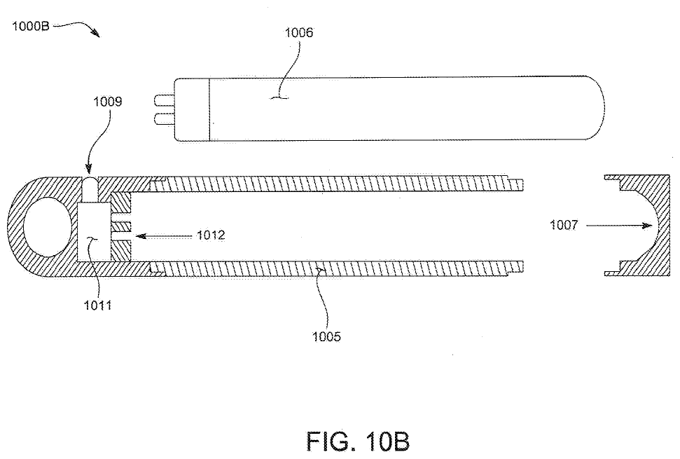

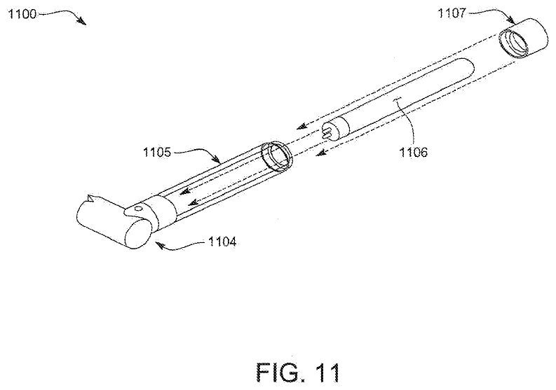

FIG. 10A is view of the ultraviolet lamp assembly of the self-disinfecting door handle according to an embodiment of the present disclosure. There is shown a UV bulb 1006 arranged in a UV lamp 1005.

FIG. 10B is an exploded view of the ultraviolet lamp assembly of the self-disinfecting door handle shown in FIG. 10A according to an embodiment of the present disclosure. The UV bulb 1006 is shown apart from the UV lamp 1005. A socket 1012 receives the UB bulb 1006 and provides an electrical connection. The lamp end 1007 provides protection and support to the opposite end of the bulb 1006. The socket 1012 in turn is connected to a s component including an electrode 1011. A wire 1009 that is attached to the power source in the latch assembly (not shown in FIG. 101B) is also shown.

FIG. 11 is an exploded view of some components of the door handle according to an embodiment of the present disclosure. The lamp assembly 1104 has the UV bulb 1106 included, and capped by lamp end 1107. The UV passable material 1105 is also shown. The material used for the handle can be translucent or transparent to various ranges of UV light. The discussion of FIG. 1 discloses some non-limiting examples of materials that may be used to construct a grip portion of the door handle 1104.

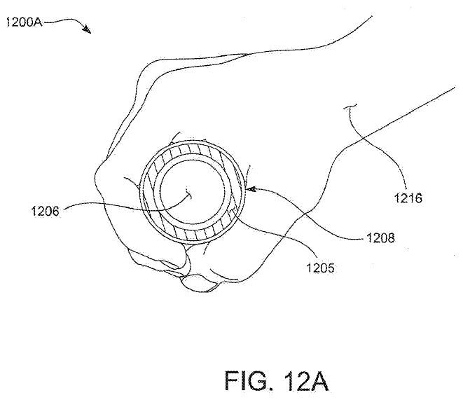

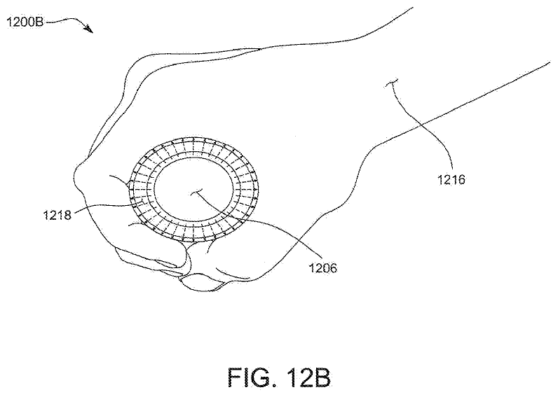

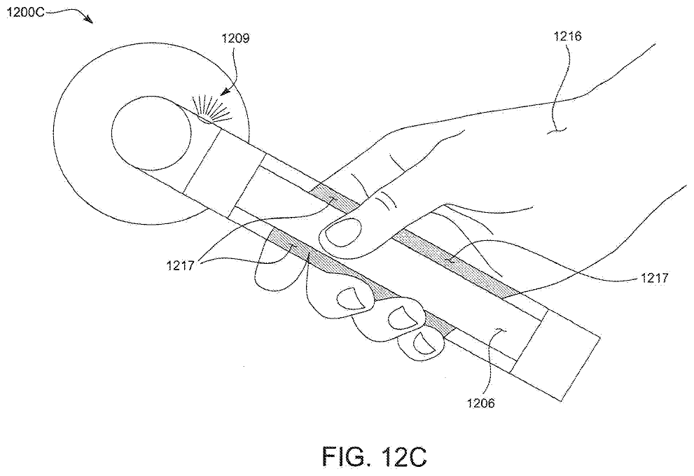

FIG. 12A is a side view 1200A of a self-disinfecting door handle grasped to open/close a door according to an embodiment of the present disclosure. It is shown the bulb 1206 is substantially concentric to the UV lamp 1205. The outer layer of translucent or transparent UV passable material 1208 is what comes into contact with a user's hand 1216 who grasped the handle.

FIG. 12B shows aside view 1200B of plurality of projections of the ultraviolet light from a self-disinfecting door handle grasped to open/close a door according to an embodiment of the present disclosure. The arrows represent the US light being emitted from UV bulb 1206. As previously discussed, the UV bulb 1206 in this embodiment is a ballast-free construction. In addition, or alternatively, a series of LEDs may be arranged with the housing of the UV bulb 1206.

The UV bulb 1206 may turn on for a predetermined period of time after contact of the door handle by a user. Such predetermined time can be, for example, 15 minutes for strong disinfection, or five minutes for less disinfection. It is also understood that the power of the UV light will also be a factor in determining a length of the predetermined time interval that the UV light is projected at the surface of the handle. Alternatively, the UV light may be projected at the surface of the handle at predetermined time intervals (e.g., every half hour). Contact with the door handle may reset the scheduled intervals of disinfection. The UV light can be on continuously for a period of time for disinfection, and may turn off when touched by a user to prevent exposing someone's hand to UV light.

FIG. 12C is a front view 1200C of a self-disinfecting door handle grasped to open a door according to an embodiment of the present disclosure. The user's hand 1216 grips a portion 1217 of the handle. The sensor 1209 can detect a user has grabbed the handle and activate the bulb 1206 for a disinfection cycle.

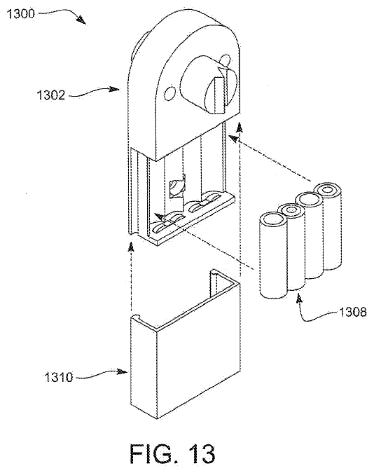

FIG. 13 is a partially exploded view 1300 of the latch assembly 1302 according to an embodiment of the present disclosure. It is shown a cover 1310 protects batteries 1308 that can be arranged in the latch assembly 1302 to power the UV lamps in the door handles. While in this embodiment, the batteries 1308 are lithium-ion, it is understood that the appended claims are not limited to a particular type of battery, or a quantity of cells shown in FIG. 13, which is provided for illustrative purposes.

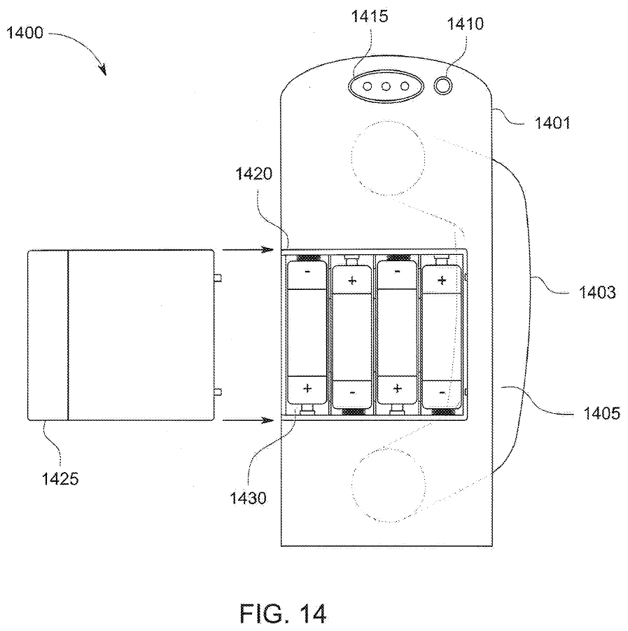

FIG. 14 is a perspective view 1400 of a self-disinfecting door pull 1401 according to an embodiment of the present disclosure. The door pulls 1401 is shown in an elongated C-shape. However, it is to be understood that any geometric shape door pull may have the self-disinfecting properties as discussed in the above embodiments, including but not in any way limited to knob-shaped. The door pull 1401 shown in FIG. 14 may include a door grip 1403, and a UV LED lamp 1405 arranged in the door grip 1403. A safety motion sensor 1410 that detects a person approaching and deactivates the UV Led lamp 1405 for a period of time while someone grabs the door grip 1403. The indicator lights 1415 may function similarly to the discussion of the indicator lights 111 shown in FIG. 1.

The door pull 1401 also includes a battery compartment 1420 and a battery cover 1425. The battery compartment may house one or more batteries 1430, including but not in any way limited to lithium batteries. The batteries 1430 optionally may be user-supplied.

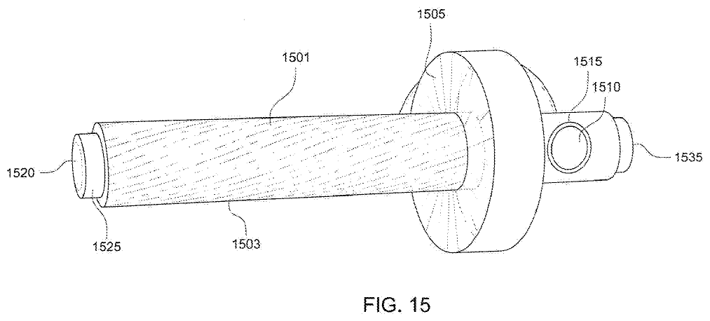

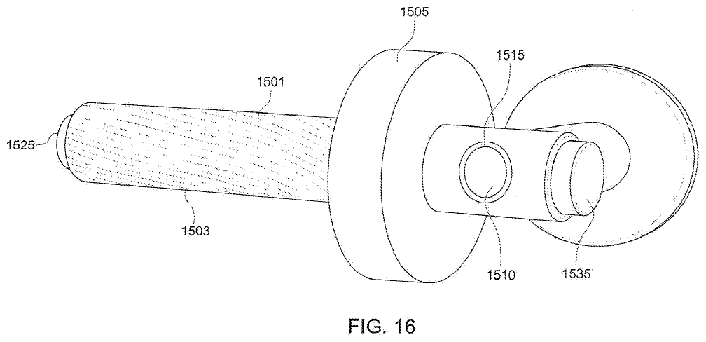

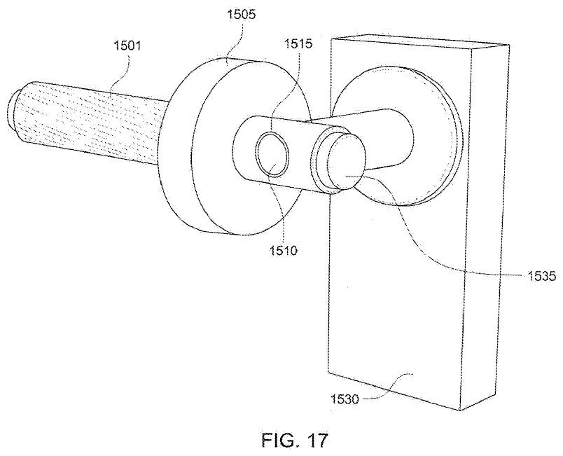

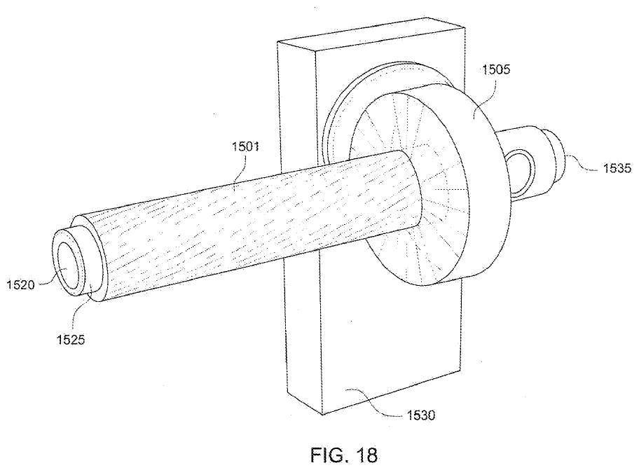

FIGS. 15-18 a self-disinfecting door handle 1501 according to an embodiment of the present disclosure. The door handle 1501 is shown in a cylindrical shape. However, it is to be understood that any geometric shape door handle may have the self-disinfecting properties as discussed in the above embodiments, including but not in any way limited to knob-shaped. The door handle 1501 shown in FIGS. 15-18 may include a door grip 1503, and a UV LED lamp 1505 arranged adjacent with door grip 1503. A safety motion sensor 1510 that detects a person approaching and deactivates the UV Led lamp 1505 for a period of time while someone grabs the door grip 1503. The indicator lights 1515 may function similarly to the discussion of the indicator lights 111 shown in FIG. 1. A fingerprint scanner 1520 is shown in the door handle 1501.

The door handle 1501 also includes a battery compartment. The battery compartment may be located inside the door handle 1501 and comprise a battery cover 1525. The battery compartment may house one or more batteries, including but not in any way limited to lithium batteries. The batteries optionally may be user-supplied. The battery compartment may be located inside compartment 1530. The compartment 1530 may also contain electronic chips such as, for example, a computer processing unit (CPU).

According to some embodiments, the lamp 1505 may be circularly shaped and comprise a through aperture configured to accommodate the door handle 1501. According to some embodiments, the UV LED lamp 1505 may be donut shaped and comprise a through aperture configured to accommodate the door handle 1501. However, it is to be understood that the UV LED lamp 1505 may comprise any geometric shape, including but not in any way limited to rectangular.

According to some embodiments, the UV LED lamp 1505 may comprise glass chambers with UV module or strip LED light. According to some embodiments, the UV LED lamp 1505 may comprise UV lamp and/or LED light.

The door handle 1501 may also include a locking button 1535 for locking the door.

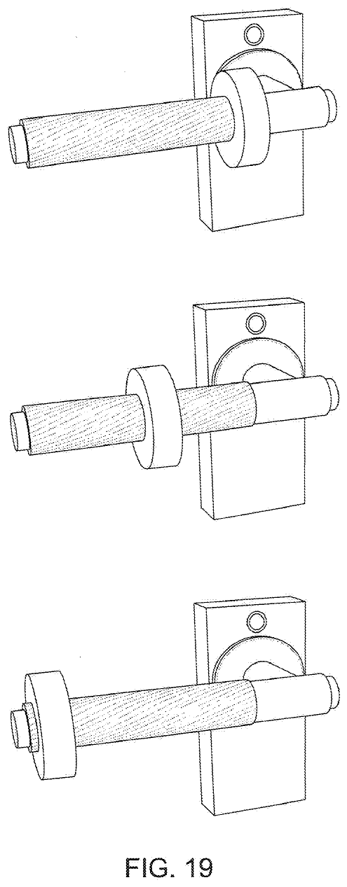

FIG. 19 is a perspective view of a self-disinfecting door handle in various states of operation to depict how the outer ring moves along the handle to safely disinfect the surface without exposing anyone in the room to harmful UV radiation.

### CONCLUSION

The descriptions of the various embodiments of the present teachings have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

The components, operations, steps, features, objects, benefits, and advantages that have been discussed herein are merely illustrative. None of them, nor the discussions relating to them, are intended to limit the scope of protection. While various advantages have been discussed herein, it will be understood that not all embodiments necessarily include all advantages. Unless otherwise stated, all measurements, values, ratings, positions, magnitudes, sizes, and other specifications that are set forth in this specification, including in the claims that follow, are approximate, not exact. They are intended to have a reasonable range that is consistent with the functions to which they relate and with what is customary in the art to which they pertain.

Numerous other embodiments of the disclosure are also contemplated. There are also embodiments of the disclosure in which the components and/or operations are arranged and/or ordered differently than described and shown herein.

It will be understood that the terms and expressions used herein have the ordinary meaning as is accorded to such terms and expressions with respect to their corresponding respective areas of inquiry and study except where specific meanings have otherwise been set forth herein. Relational terms such as first and second and the like may be used solely to distinguish one entity or action from another without necessarily requiring or implying any such actual relationship or order between such entities or actions. The terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element proceeded by “a” or “an” does not, without further constraints, preclude the existence of additional identical elements in the process, method, article, or apparatus that comprises the element.

What is claimed is:

1. A self-disinfecting door handle device, comprising:
a first door handle having a first grip portion constructed of an ultraviolet (UV) light passable material having at least a semi-transparency for passage of an ultraviolet light;
a latch assembly rotatably connected to the first door handle, the latch assembly includes a latch that is configured to retract according to a rotated position of the first door handle; and
a UV lamp arranged within the first door handle, wherein the UV lamp includes a connector configured to electrically connect to a UV light source that emits a UV light toward a surface of at least the first grip portion of the first door handle.

2. The device according to claim 1, wherein the UV map emits light having a wavelength of 222 nm.

3. The device according to claim 1, further comprising the latch assembly includes a battery compartment electrically connected to the UV lamp; and
wherein the UV lamp receives power from a power source arranged in the battery compartment selected from the group consisting of lithium ion, sodium ion, potassium ion, lithium-sulfur, sodium sulfur, and potassium sulfur.

4. The device according to claim 1, further comprising the UV light source being electrically connected to the UV lamp, wherein the first door handle comprises a substantially tubular shape configured to enclose the UV light source, and
wherein the UV light source comprises a plug and play UV tube.

5. The device according to claim 4, wherein the UV light source comprises a ballast-free UV light source.

6. The device according to claim 4, wherein the UV light source comprises one or more UV light emitting diodes (LED).

7. The device according to claim 1, further comprising a second door handle rotatably connected to an opposite side of the latch assembly, the second door handle having a second grip portion constructed of an ultraviolet (UV) light passable material having at least a semi-transparency for passage of an ultraviolet light.

8. The device according to claim 7, wherein at least the first door handle and/or the second door handle is coated with an anti-microbial substance.

9. The device according to claim 6, wherein at least the first door handle and/or the second door handle is constructed of an anti-microbial material.

10. The device according to claim 4, wherein the UV light source emits UVC light toward an exterior surface of the first grip portion of the first door handle.

11. The device according to claim 4, wherein the UV light source emits one of FAR-UV light of NEAR-UV light toward an exterior surface of the first grip portion of the first door handle.

12. The device according to claim 1, further comprising a sensor arranged in the first door handle configured to detect when the first grip portion handle is contacted by a user.

13. The device according to claim 12, wherein the UV lamp activates the UV light source based on whether the sensor detects a contact of the first grip portion.

14. A self-disinfecting door pull, comprising:
a handgrip configured for attachment to a door;
a sensor configured to detect contact with an exterior surface of the handgrip; and
an ultra-violet (UV) light source arranged to emit UV light at the exterior surface of the handgrip.

15. The self-disinfecting door pull of claim 14, when an exterior of the handgrip is constructed of an anti-microbial material and/or coated with an anti-microbial agent.

16. The self-disinfecting door pull of claim 14, further comprising a motion sensor configured to detect at least a body portion within a predetermined distance of the door pull.

17. The self-disinfecting door pull of claim 16, wherein the UV light comprises a UV LED light arranged external to the door pull.

18. The self-disinfecting door pull of claim 14, further comprising the UV light source comprises a ballast-free UV light source arranged within the handgrip, and wherein the handgrip is constructed of a UV-passable material.

19. The self-disinfecting door pull of claim 18, wherein the UV light source is configured to emit UV light at a surface of the handgrip for a predetermined period of time after the sensor detects contact with the exterior surface of the tube.

20. The self-disinfecting door pull of claim 17, wherein the UV light a battery compartment for housing a power source.

21. The self-disinfecting door pull of claim 14, further comprising:
a latch assembly rotatably connected to the handgrip, and
wherein the latch assembly includes a battery compartment configured for receiving a lithium battery that provides power to the UV light source, and
wherein the UV light source is configured to emit UV light at the handgrip for predetermined intervals of time.

22. A self-disinfecting door handle device, comprising:
an outer ring on the door handle with a 254 nm UV germicidal light inside of the outer ring;
an inner magnet that is mounted on a rail system in the interior of the handle; and
a power source located in the doorplate.

23. The self-disinfecting door handle of claim 21, wherein the outer ring seals the UV light inside the outer ring to limit exposure to only the handle and not people located in the room.

24. The self-disinfecting door handle of claim 21, wherein the outer ring is moved along the exterior of the door handle based on an inner magnet that is mounted on a rail system in the interior of the handle.

25. The self-disinfecting door handle of claim 21, wherein the end of the handle has a cap or ridge which prevents the outer ring from accidentally coming off.

26. The self-disinfecting door handle of claim 21, wherein the internal rail system is powered directly from the power source located in the door plate and the outer ring is powered by a wirelessly rechargeable battery.

27. The self-disinfecting door handle of claim 21, wherein the interior rail system is powered directly by wires from the battery located inside the doorplate. The exterior ring where the UV light is located has a battery that is wirelessly charged while not in use and located in the interior position.
